(19) United States
(12) Patent Application Publication
(10) Pub. No.: US 2022/0401381 A1
(43) Pub. Date: Dec. 22, 2022

(54) PERIPHERALLY ACTING CANNABIDIOL (CBD)-CONTAINING COMPOUNDS AND USES THEREOF FOR ENHANCING FEMALE SEXUAL FUNCTION OR TREATING FEMALE SEXUAL DISORDERS

(71) Applicant: Vella Bioscience, Inc., Worcester, MA (US)

(72) Inventors: Harin Padma-Nathan, Los Angeles, CA (US); Michael Frid, Medford, MA (US)

(21) Appl. No.: 17/775,287

(22) PCT Filed: Nov. 3, 2020
(86) PCT No.: PCT/US20/58722

§ 371 Date: Jul. 6, 2022

Related U.S. Application Data
(60) Provisional application No. 62/972,288, filed on Feb. 10, 2020.
(60) Provisional application No. 62/932,762, filed on Nov. 8, 2019.

(51) Int. Cl. A61K 31/05; A61K 9/00; A61K 9/127; A61K 31/496; A61K 31/4985; A61K 31/53; A61K 31/519; A61K 31/5575; A61K 31/472; A61K 31/506; A61K 31/417; A61K 38/12; A61P 15/00; A61F 6/04

(52) U.S. Cl. CPC A61K 31/05; A61K 9/0034; A61K 9/127; A61K 31/496; A61K 31/4985; A61K 31/53; A61K 31/519; A61K 31/5575; A61K 31/472; A61K 31/506; A61K 31/417; A61K 38/12; A61P 15/00; A61F 6/04

(57) ABSTRACT

The present disclosure provides peripherally acting cannabidiol (CBD)-containing compositions and methods of using thereof for treating or enhancing female sexual function. In some embodiments, the compositions are provided in the form of a lotion, containing highly CBD-loaded liposomes, which is applied to female genitalia shortly prior to sexual activity.

### PRIORITY

This application claims priority to U.S. Provisional Patent Applications Nos. 62/932,762, filed on Nov. 8, 2019, and 62/972,288, filed on Feb. 20, 2020, both of which are hereby incorporated reference.

### FIELD OF THE INVENTION

The invention relates to compositions and methods containing hemp and/or cannabis-derived cannabinoid(s) for topical use in enhancing female sexual function and treating or ameliorating certain female sexual disorders.

### BACKGROUND

The endocannabinoid system (ECS) is a major neuromodulatory regulatory system found in the central nervous system and in select peripheral nerves and organs. It is made up of cannabinoid (CB1 and CB2) receptors, their endogenous ligands (endocannabinoids: anandamide (AEA) and 2-arachidonoylglycerol), proteins involved in the synthesis and breakdown of endocannabinoids, and the intracellular signaling pathways affected by cannabinoids. CB1 and CB2 receptors are G-protein-coupled receptors that serve as the primary site of action for cannabinoids. The cannabinoid receptors differ in their distribution. CB1 receptors are found throughout the central nervous system and some peripheral tissues. Cannabinoid receptors in the CNS are found in the hypothalamus, hippocampus, amygdala, cerebral cortex, parts of the basal ganglia, and cerebellum. CB1 and CB2 receptors are presynaptic receptors that results in inhibition of neurotransmitter release when activated. CB1 receptors are located in the axon terminals of GABAergic, dopaminergic, adrenergic, glutamatergic, cholinergic and some serotonergic neurons, particularly in these primitive portions of the limbic system that control, among other things, sexual behavior. Peripherally, CB2 cannabinoid receptors are found in organs responsible for producing sex hormones. They have also been found in the ovaries, uterus, bladder, penile corpora and the testes. To date, the female vagina and clitoris have not been examined for or identified to have CB receptors.

Δ⁹-(delta 9)-tetrahydrocannabinol (THC) was the first cannabinoid identified from the Cannabis sativa plant and characterized in the 1960's by Mechoulam and associates. It is a potent sedative-hypnotic. Subsequently, over a hundred cannabinoids, including cannabidiol, a non-psychoactive cannabinoid, have been identified. Cannabidiol has been attributed to have many potential pharmacological benefits in pain, inflammation and, most recently, in infantile seizure disorders. It recently received FDA approval for the treatment of Dravet syndrome and Lennox-Gastaut syndrome. (Davinsky, O et al, N Engl J Med 2017; 376: 2011-2020; Thiele, EA et al, Lancet 201; 391(10125): 1085-1096)

The role of the hypothalamic pituitary axis on female sex hormones and thus female sexual function has been long established. The neurohormonal aspects of male and female sexual desire or interest are driven by androgens. The endocannabinoid system appears to be inhibitory to sexual responses in animals. Levels of anandamide (AEA) and 2-arachidonoylglycerol, the endocannabinoids, are lowered in response to sexual stimulation. Delta-9 tetrahydrocannabinol (THC) appears to blunt the activation of hormones that modulate female sexual responses in animal and human studies. However, exogenous Δ⁹-tetrahydrocannabinol (THC), in the presence of an intact hormonal axis, produces female rat lordosis (a sexual receptivity posture) at lower doses. This is believed to be an entirely central nervous system effect and not a peripheral response. While CB1 and CB2 receptors for endocannabinoids exist peripherally, the actions of THC on sexual function are believed to be central in action on the dopaminergic and serotonergic pathways of the limbic system (hypothalamic ventral tegmental area and nucleus accumbens). Pharmacologic doses of Δ⁹-tetrahydrocannabinol appear to augment these serotonergic and dopaminergic pathways. (Lynn, B. et al: J Sex Med, The Relationship between Marijuana Use and Prior to Sex and Sexual Function in Women. 2019; 1e10)

In contrast, sexual arousal is a peripheral genital process. Female and male sexual arousal, as evidenced by clitoral engorgement and vaginal lubrication or penile erection, respectively, are regulated by the tone of the smooth muscle of the clitoris and vagina and by the tone of the smooth muscle of the corpora cavernosa and corpus spongiosum. CB1 and CB2 receptors have been identified in the cavernosal endothelium and smooth muscle of male primates and humans. Gratzke, C et al: Localization and function of cannabinoid receptors in the corpus cavernosum: basis for modulation of nitric oxide synthase nerve activity (Eur Urol 2010 57(2): 342-349. Sexual arousal in males may be related to nitrergic pathways in the corpus cavernosum. However, despite identification of CB2 receptors in ovaries and endometrial tissue, there have been no studies to confirm the presence of vaginal or clitoral CB receptors in animal or human tissue and thus no prior evidence to support a role for cannabinoids, such as cannabidiol, to effect arousal through a peripheral pharmacologic action on this smooth muscle. To date, there have been no examinations of a direct pharmacologic effect of any individual cannabinoid on vaginal or clitoral smooth muscle function, such as the standard studies in rat and human vaginal tissue to demonstrate a pharmacologic impact on smooth muscle and its related endothelium which are usually performed in isolated tissue organ baths, examining the dose or concentration related relaxations responses (lowered isometric tension) in precontracted smooth muscle tissue. Munarriz R, Traish A, Goldstein I: A review of the physiology and pharmacology of peripheral (vaginal and clitoral) female genital arousal in the animal model. (J Urol. 2003 Aug; 170 (2 Pt 2): 540-4).

The results of studies in men have been equivocal as to whether cannabis facilitates or inhibits sexual function, but recent large population studies indicate that, in low doses, cannabis produces sexual facilitatory effects in men. In contrast, the results in women have almost consistently suggested a pro-sexual effect. A recent, large population-based study by Sun et al, to elucidate whether or not a relationship between marijuana (inhaled cannabis) use and sexual frequency exists, utilized a nationally representative sample of reproductive-age men and women. They analyzed data from the National Survey of Family Growth, a nationally representative cross-sectional survey. Investigators used a multivariable model, controlling for demographic and socioeconomic characteristics, to evaluate whether a relationship between marijuana use and sexual frequency exists. They examined sexual frequency within the 4 weeks preceding the survey administration related to marijuana use and frequency in the year preceding survey administration in 28,176 women (average age=29.9 years) and 22,943 men (average age=29.5). They found that female monthly (incidence rate ratio [IRR]=1.34, 95% CI=1.07-1.68, P=0.012), weekly (IRR=1.36, 95% CI=1.15-1.60, P<.001), and daily (IRR=1.16, 95% CI=1.01-1.32, P=0.035) marijuana users had significantly higher sexual frequency compared with never users. And male weekly (IRR=1.22, 95% CI=1.06-1.41, P=0.006) and daily (IRR=1.36, 95% CI=1.21-1.53, P<0.001) users had significantly higher sexual frequency compared with never users. An overall trend for men (IRR=1.08, 95% CI=1.05-1.11, P<0.001) and women (IRR=1.07, 95% CI=1.04-1.10, P<0.001) was identified showing that higher marijuana use was associated with increased coital frequency. They concluded that marijuana use is independently associated with increased sexual frequency and does not appear to impair sexual function. Since the participants appeared to only consume inhaled cannabis, it is difficult to attribute the outcomes to a specific cannabinoid. However, it is clear that cannabis is a pro-sexual product, particularly when consumed in low doses. (Sun A J, et al: J Sex Med 2017; 14: 1342-1347). Cannabidiol is of potential interest in sexual augmentation since it has demonstrated some peripheral activity and would be amendable to local delivery. The recent marketing of topical female sexual products containing cannabis extracts in coconut oil have not examined actual outcomes utilizing physiologic (vaginal photoplethysmography) measures or any at-home, real world outcomes utilizing such measures as the Female Sexual Function Index questionnaire (Rosen, Ret al: (2000) J Sex & Marital Ther 26(2); 26: 2, 191-208). The latter has served as the primary outcome measure in pharmacologic clinical trials in female sexual dysfunction, FDA-registered drug trials. The only captured data from the use of these products are anecdotal testimonials and these seem to strongly indicate the presence of a placebo effect or systemic effects (see www.foriawellness.com/products/foria-awaken). This is not surprising since it is questionable whether or not the highly lipophilic cannabinoid extract or cannabidiol formulated in coconut oil are absorbed through the vaginal mucosa and penetrate to the sites of action, that is, whether or not cannabinoid formulations in coconut oil or other water-immiscible lipids and/or triglycerides are able to deliver adequate doses of cannabinoids to the appropriate underlying smooth muscle and endothelium. Additionally, these agents, particularly those in coconut oil or those that are not encapsulated, would put in danger the practice of safe sex since these agents are known to dramatically compromise the integrity of latex condoms.

Female Sexual Disorders are defined in The Diagnostic and Statistical Manual of Mental Disorders-5 (“DSM-5”) and include Sexual Interest/Arousal Disorder and Female Orgasmic Disorder (as well as Genito Pelvic Pain/Penetration Disorder). Attempts to improve arousal in women utilizing the same pharmacologic agents (e.g., sildenafil, tadalafil, prostaglandins) that have been successfully utilized in men with erectile dysfunction (ED) have produced no statistically significant effects in women, even when selective subpopulations have been examined. Female sexual disorders or dysfunctions (ESDs) have a high prevalence, with upwards of 40% of women complaining of one or more of such dysfunctions, and these are often associated with secondary effects on quality of life such as relationships, as well as anxiety, mood and self-esteem. The two currently approved treatments for FSD are for desire disorders and are both centrally acting drugs. No effective peripherally acting agents have been approved by regulatory agencies (e.g. US FDA) for augmentation or enhancement of sexual function in otherwise normal or intact women (from a sexual function perspective) or for the treatment of interest/arousal disorders or orgasmic disorders in women (Diagnostic and Statistical Manual of Mental Disorders: DSM-5. 5th ed., 2013).

Thus, there remains a need to provide novel composition and methods for improving female sexual function and treating or ameliorating certain sexual disorders in women.

### SUMMARY OF THE INVENTION

The present disclosure provides peripherally acting cannabidiol (CBD)-containing compositions and methods of using thereof for treating or enhancing female sexual function. In some embodiments, the compositions are provided in the form of a lotion, containing highly CBD-loaded multilamellar liposomes (or optionally, multilamellar and unilamellar liposomes, or a mixture thereof), which is applied to the female genitalia shortly prior to sexual activity. In some embodiments, the compositions are provided in the form of a lotion, containing highly CBD-loaded liposomes (e.g., multilamellar, unilamellar or a mixture thereof), which is applied to the female genitalia shortly prior to sexual activity.

The present invention is based, at least in part, on the following:

1. Our demonstration of an original, unexpected pharmacologic effect of cannabidiol to relax precontracted rat vaginal smooth muscle in a dose-dependent manner in normal and oophorectomized rats;
2. Our unique formulation of nanoencapsulated, large liposomes loaded with CBD for delivery such that the liposomes that increases payload, bioavailability/efficacy and provides condom compatibility for safe sexual activity; and
3. A gold standard, at-home open-label study of the invention when utilized in an on-demand basis by premenopausal and post-menopausal women with intact desire and orgasm capability.

One aspect of the invention may further be based on a prediction that the application of the invention would lead to increased vaginal blood flow in response to such application, wherein the blood flow is measured, for example, by standard vaginal photoplethysmography as a pre-requisite to orgasmic state (see, e.g., Example 8).

In some embodiments, the invention features a method of improving female sexual function. In other embodiments, the invention features treating a female sexual disorder such as, for example, Sexual Interest/Arousal Disorder (SIAD) and Female Orgasmic Disorder.

In general, the methods of the invention employ topical composition formulated with liposomes that comprise cannabidiol (CBD) and, optionally, one or more additional cannabinoids. In the methods of the invention, such a composition is applied topically to a female subject's genital (arousal) area(s), to surface with absorptive mucosa, such as, for example, the vulva, the introitus, the labia minora, the clitoris and the vaginal vault.

In general, the compositions of the invention are applied either as lotion and/or as lubricant in the amount and for a period of time prior to a sexual activity such that sexual function of the subject is enhanced and/or the disorder is ameliorated during the sexual activity, as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm more often”; “greater ease of achieving orgasm”; “being more satisfied”; “higher level of sexual desire”; and “reduction in pain during sexual activity”. In some embodiments, the subjects are premenopausal, while in other embodiments, certain parameters reported by post-menopausal women are similar to those reported by premenopausal women. The composition can be applied 1-60 min prior to sexual activity, preferably, 5-40 min, more preferably 15-20 min.

In some embodiments, the concentration of CBD in the composition is from 1 mg/ml to 40 mg/ml, preferably, 5 mg/ml to 20 mg/ml, more preferably, 10 mg/ml. In general, the total amount of CBD per application is from 5 mg to 1,000 mg of CBD, preferably 10-100 mg of CBD, more preferably, 20-40 mg of CBD, most preferably 20 mg. The CBD-containing composition may be applied multiple-times, and the total dose may be subject-specific. In some embodiments, the CBD-containing composition is applied at 5-30 mins prior to the sexual activity, preferably 10-20 mins. In some embodiments, the off-set time following the application of the CBD-containing composition is 0.5-5 hrs, preferably, 1-3 hrs, more preferably 1-2 hrs.

The CBD-containing composition is compatible with latex and polyisoprene condoms, and in some embodiments, may be provided by means of a condom pre-coated with such a composition. In preferred embodiments, CBD-containing composition also comprises liposomes that comprise HSPC, ascorbic acid, sodium ascorbate, propylene glycol, polyacrylate crosspolymer-6, and water or aqueous buffer, and wherein the liposomes are provided in a homogeneous suspension. The additional cannabinoid(s) can be present in the composition, for example, such as Δ⁹-THC, Δ⁸-THC, CBD, and CBN and another cannabinoid listed in this disclosure. In some embodiments, CBD is hemp-derived and/or contains less than 0.3% THC by weight.

With respect to treatment of sexual disorders, it is expected that the subject, upon having been treated with the CBD-containing composition for 3-6 months, exhibits improvement as measured by FSFI, for example, in sexual interest/arousal domains is by 1.5-2 points, and by 1.5-2 points in the orgasm domain of the FSFI.

In yet further embodiments, a phosphodiesterase type 5 inhibitor such as, for example, sildenafil, tadalafil, vardenafil, udenafil, can be added to the CBD-containing composition since this should produce or augment the nitrergic amplification.

In another embodiment, another erectogenic smooth muscle relaxant such as for example, prostaglandin E1, papaverine, minoxidil, can be added to the CBD-containing composition.

In another embodiment, an alpha-blocker (e.g., phentolamine) can be added to the CBD-containing composition.

In another embodiment, flibanserin can be added to the CBD-containing composition to augment sexual desire.

In another embodiment, bremelanotide can be added to the CBD-containing composition to augment sexual desire.

A more detailed description of the invention follows.

### DETAILED DESCRIPTION OF THE DRAWINGS

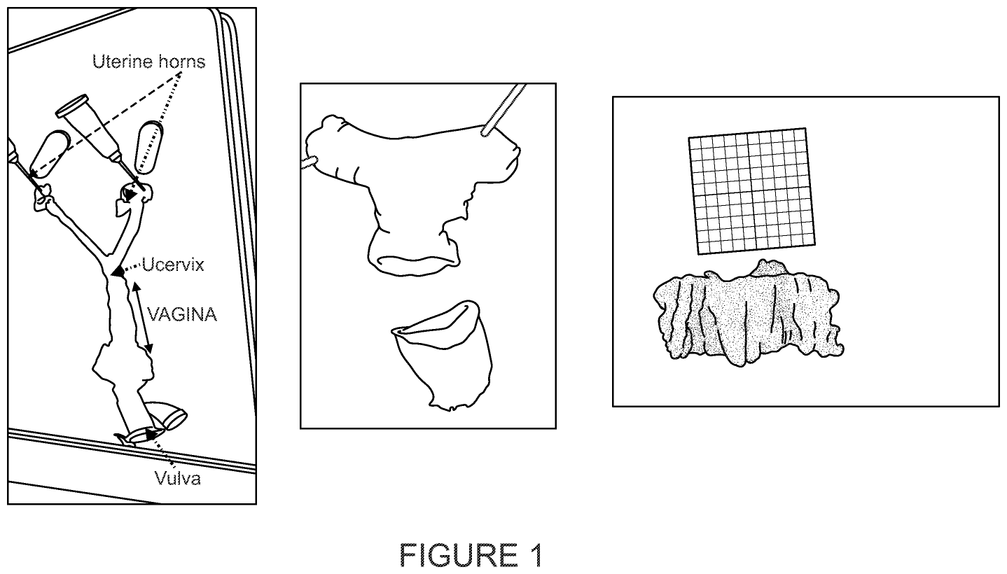

As shown in FIGS. 1, female rats either intact (n=6) or bilaterally oophorectomized (OVX) (n=6 rats); the animals were euthanized 6 weeks post OVX or sham surgery and the vagina dissected. Then, the vagina was cut into 2 halves, proximal and distal, and 4 strips max per region cut in circular direction were prepared and mounted as shown. The strips were excised from the tissue samples and then connected to force transducers for isometric tension recording.

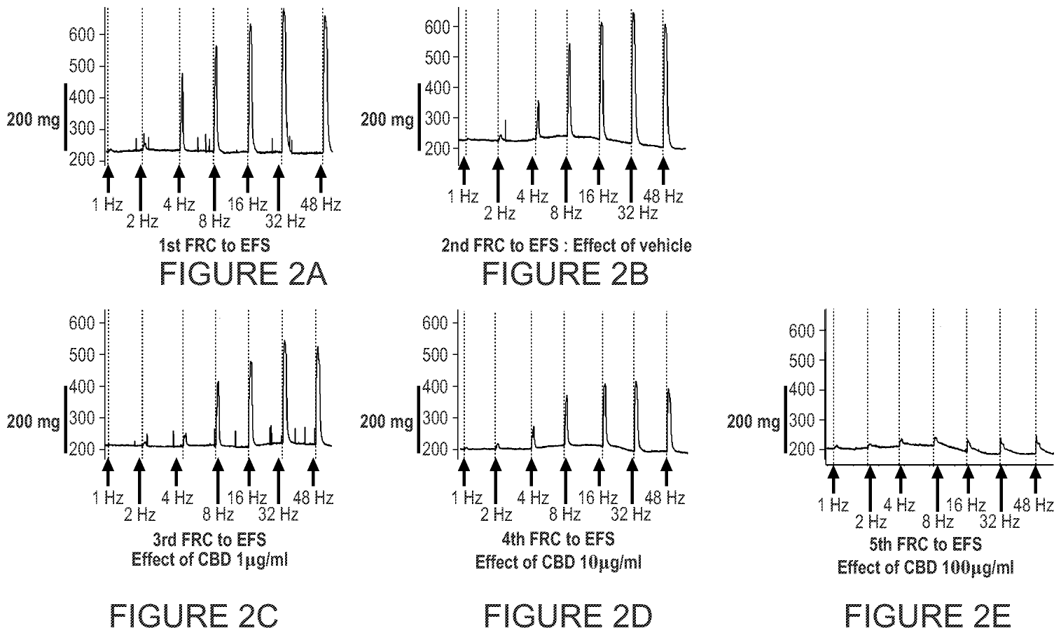

FIG. 2A shows representative tracings from individual tissues. Contractile responses of tissue to Electrical Field Stimulation (EFS)-induced contraction of vaginal strips from intact/sham rats.

FIG. 2B shows representative tracings from individual tissues. Effect of vehicle (no active drug) on EFS-induced contraction of vaginal strips from intact/sham rats.

FIGS. 2C shows representative tracings from individual tissues. Effect of CBD at 1 μg/ml on EFS-induced contraction of vaginal strips from intact/sham rats.

FIG. 2D shows representative tracings from individual tissues. Effect of CBD at 10 μg/ml on EFS-induced contraction of vaginal strips from intact/sham rats.

FIGS. 2E shows representative tracings from individual tissues. CBD at 100 μg/ml on EFS-induced contraction of vaginal strips from intact/sham rats.

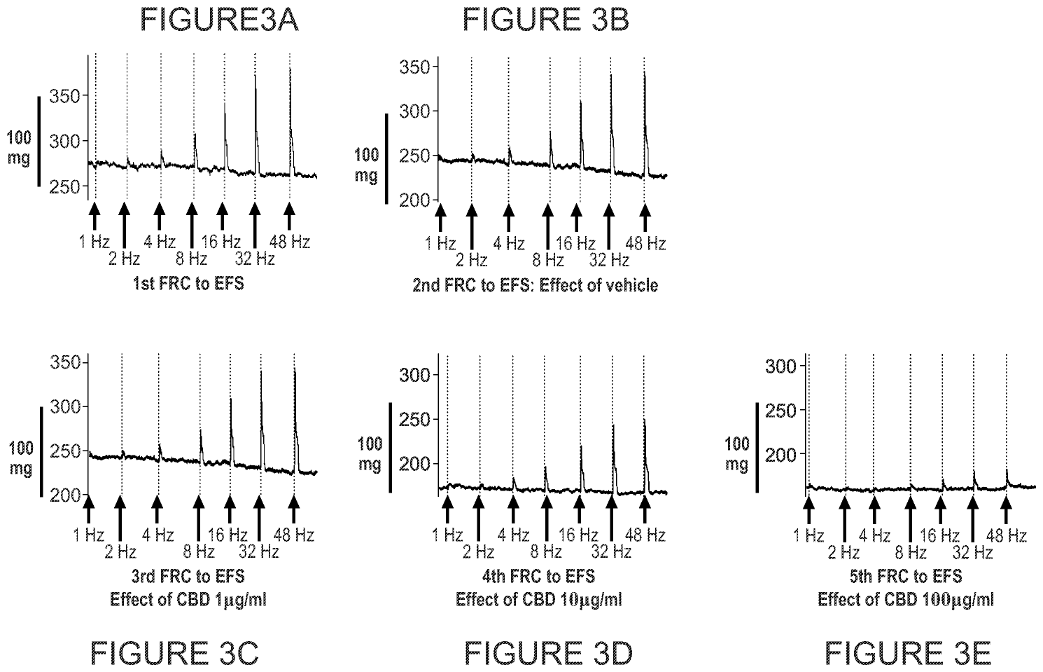

FIG. 3A-3E show representative tracings from individual tissues Effect of vehicle and CBD at 1, 10 and 100 μg/ml on EFS-induced contraction of vaginal strips from OVX rats.

FIG. 3A shows representative tracings from individual tissues. Contractile responses of tissue to EFS-induced contraction of vaginal strips from OVX rats.

FIG. 3B shows representative tracings from individual tissues. Effect of vehicle (no active drug) on EFS-induced contraction of vaginal strips from OVX rats.

FIG. 3C shows representative tracings from individual tissues. Effect of CBD at 1 μg/ml on EFS-induced contraction of vaginal strips from OVX rats.

FIG. 3D shows representative tracings from individual tissues. Effect CBD at 10 μg/ml on EFS-induced contraction of vaginal strips from OVX rats.

FIG. 3E shows representative tracings from individual tissues. Effect of CBD at 100 μg/ml on EFS-induced contraction of vaginal strips from OVX rats.

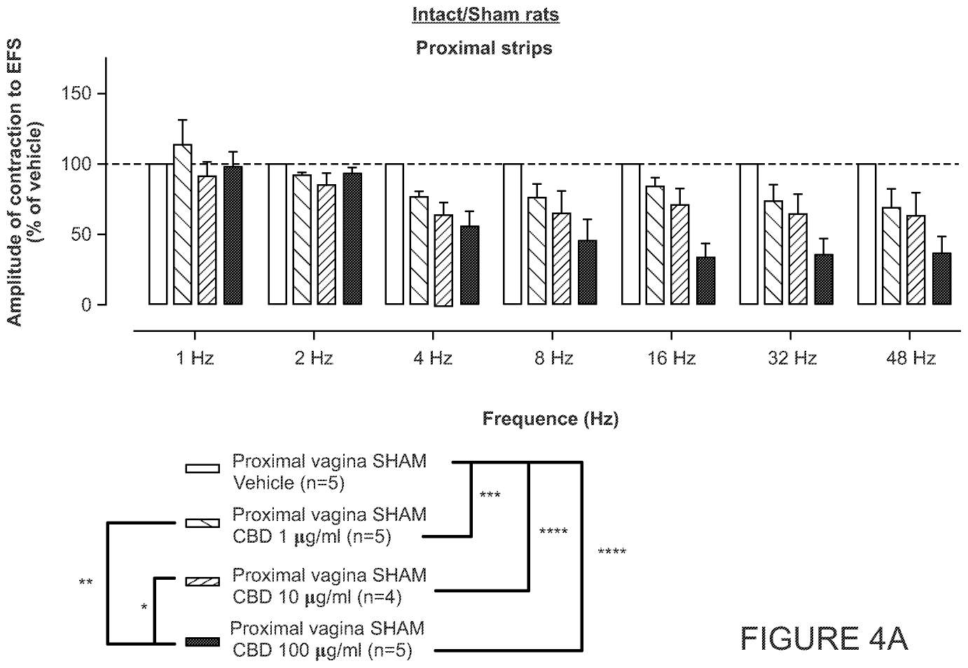

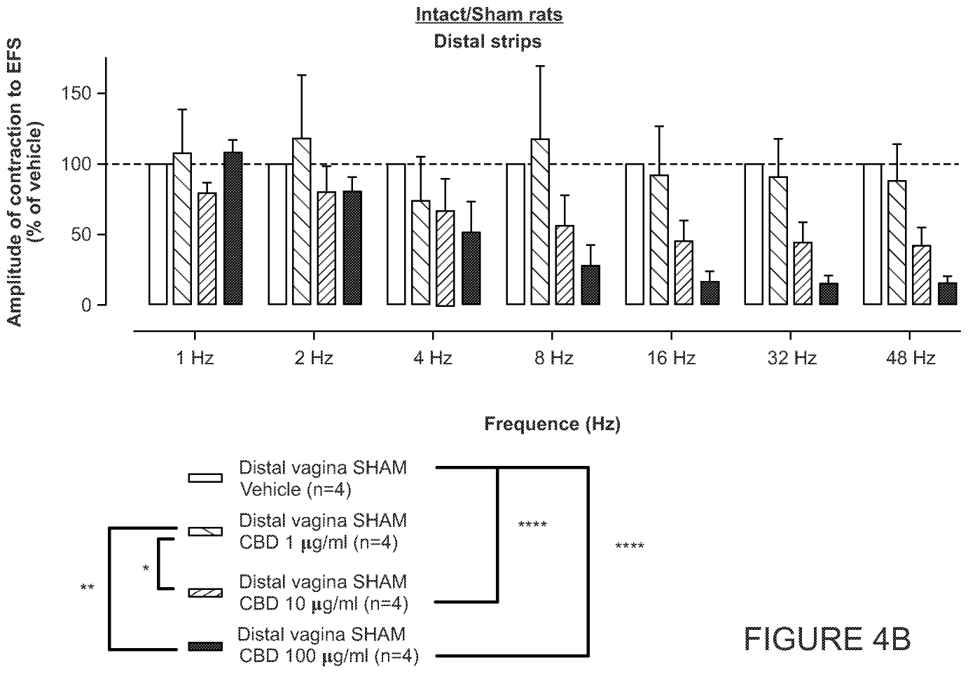

FIG. 4A demonstrates that CBD has a statistically significant, unexpected, peripheral, dose-dependent pharmacological relaxant effect on intact/sham rat proximal vaginal smooth muscle tissue.

FIG. 4B demonstrates that CBD has a statistically significant, unexpected, peripheral, dose-dependent pharmacological relaxant effect on intact/sham rat distal vaginal smooth muscle tissue.

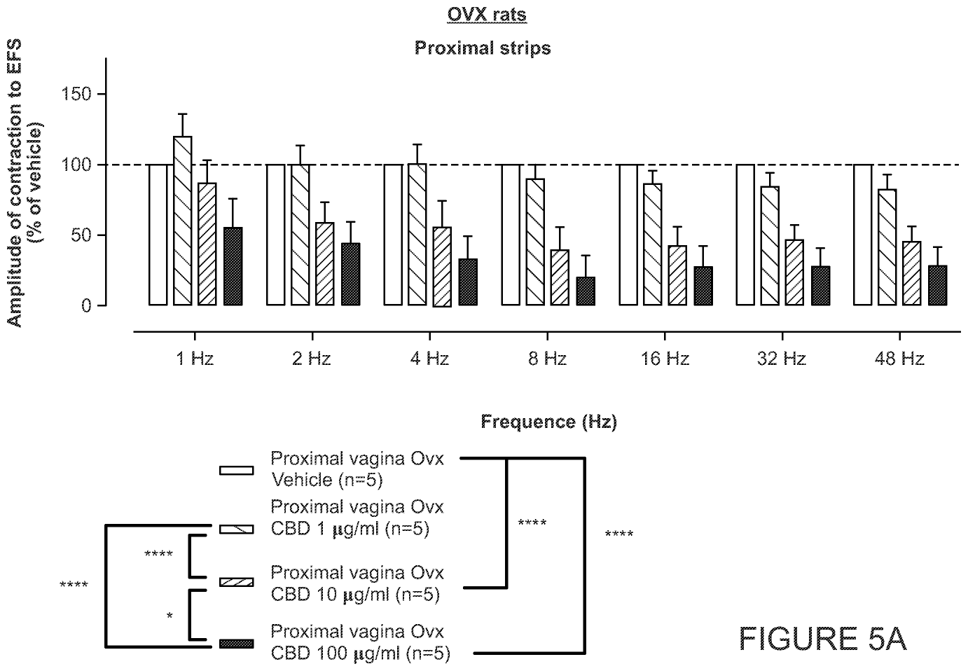

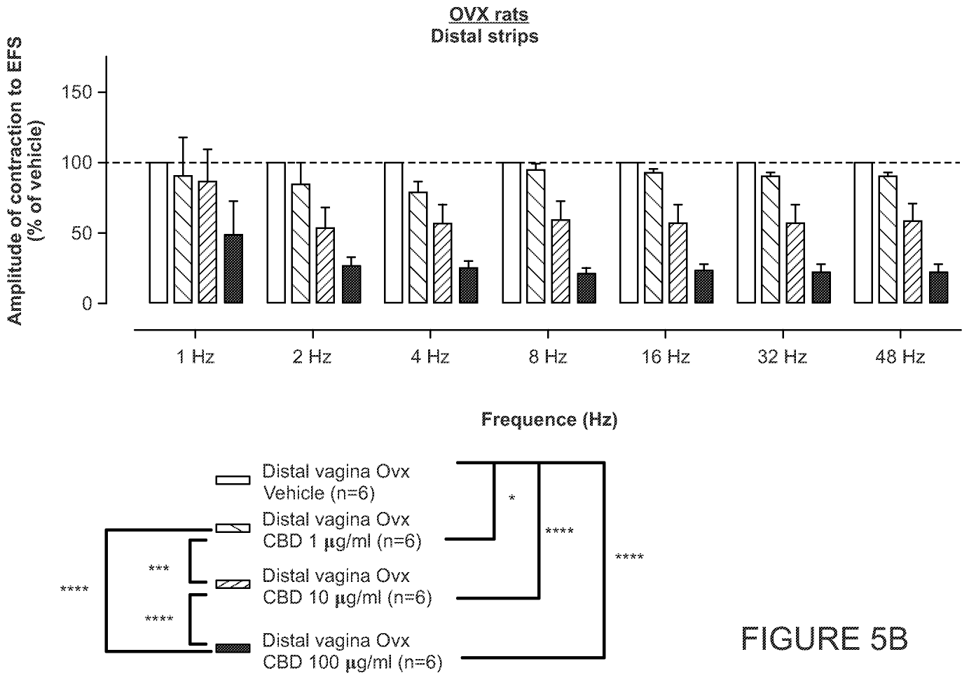

FIG. 5A demonstrates that CBD has a statistically significant, unexpected, peripheral, dose-dependent pharmacological relaxant effect on OVX rat proximal vaginal smooth muscle tissue.

FIG. 5B demonstrates that the effects of CBD have a statistically significant, unexpected, peripheral, dose-dependent pharmacological relaxant effect on OVX rat distal vaginal smooth muscle tissue.

### DETAILED DESCRIPTION OF THE INVENTION

The present disclosure provides peripherally acting cannabidiol (CBD)-containing compositions and methods of using thereof for treating or enhancing female sexual function. In some embodiments, the compositions are provided in the form of a lotion, containing highly CBD-loaded liposomes, which is applied to female genitals shortly prior to sexual activity. In some embodiments, the compositions are provided in the form of a lotion, containing highly CBD-loaded liposomes (multilamellar, unilamellar, or a mixture thereof), which is applied to female genitals shortly prior to sexual activity.

In some embodiments, the invention features a method of improving female sexual function. In other embodiments, the invention features treating a female sexual disorder such as, for example, Sexual Interest/Arousal Disorder (SIAD) and Female Orgasmic Disorder.

In general, the methods of the invention employ topical composition formulated with liposomes that comprise a cannabidiol (CBD) and, optionally, one or more additional cannabinoids. In the methods of the invention, such a composition is applied topically to a female subject's genital (arousal) area(s), to surface with absorptive mucosa, such as, for example, the introitus, the labia minora, the clitoris and the vaginal vault.

In general, the compositions of the invention are applied either as lotion and/or as lubricant in the amount and for a period of time prior to a sexual activity such that sexual function is of the subject is enhanced and/or the disorder is ameliorated during the sexual activity as exhibited by either objective parameters (vaginal and clitoral smooth muscle relaxation and/or increased vaginal and clitoral blood flow) or improvements in self-reported outcomes such as: “increased lubrication/wetness during sexual activity”; “reaching orgasm more often”; “greater ease of achieving orgasm”; “being more satisfied”; “higher level of sexual desire”; and “reduction in pain during sexual activity”. In some embodiments, the subjects are premenopausal, while in other embodiments, certain parameters reported by post-menopausal women are similar to those reported by premenopausal women. The composition can be applied 1-60 min prior to sexual activity, preferably, 5-40 min, more preferably 15-20 min.

In some embodiments, the concentration of CBD in the composition is from 1 mg/ml to 40 mg/ml, preferably, 5 mg/ml to 20 mg/ml, more preferably, 10 mg/ml. In general, the total amount of CBD per application is from 5 mg to 1,000 mg of CBD, preferably 10-100 mg of CBD, more preferably, 20-40 mg of CBD, most preferably 20 mg. The CBD-containing composition may be able applied multiple-times, and the total dose may be subject-specific. In some embodiments, the CBD-containing composition is applied at 5-30 mins, preferably 10-20 mins, prior to the sexual activity. In some embodiments, the off-set time following the application of the CBD-containing composition is 0.5-5 hrs, preferably, 1-3 hrs, more preferably 1-2 hrs.

The CBD-containing composition is compatible with latex and polyisoprene condoms, and in some embodiments, may be provided by means of a condom pre-coated with such a composition. In preferred embodiments, CBD-containing composition also comprises liposomes that comprise HSPC, ascorbic acid, sodium ascorbate, propylene glycol, polyacrylate crosspolymer-6, and water or aqueous buffer, and wherein the liposomes are provided in a homogeneous suspension. The additional cannabinoid(s) can be present in the composition, for example, such as Δ⁹-THC, Δ⁸-THC, CBD, and CBN and another cannabinoid listed in this disclosure. In some embodiments, CBD is hemp-derived and/or contain less 0.3% THC by weight.

With respect to treatment of sexual disorders, it is expected that the subject, upon having been treated with the CBD-containing composition for 3-6 months, exhibits improvement as measured by FSFI, for example, in sexual interest/arousal domain is by 1.5-2 points, and by 1.5-2 points in the orgasm domain of the FSFI.

In yet further embodiments, a phosphodiesterase type 5 inhibitor such as, for example, sildenafil, tadalafil, vardenafil or udenafil, can be added to the CBD-containing composition.

In another embodiment, another erectogenic smooth muscle relaxant such as for example, prostaglandin E1, papaverine, minoxidil, can be added to the CBD-containing composition.

In another embodiment, an alpha-blocker (e.g., phentolamine) can be added to the CBD-containing composition.

In another embodiment, flibanserin can be added to the CBD-containing composition to augment sexual desire.

In another embodiment, bremelanotide can be added to the CBD-containing composition to augment sexual desire.

As used herein, the term “cannabinoid” or “cannabinoids” refer to phytocannabinoids produced, in whatever quantity, by plants Cannabis sativa and Cannabis indica, which naturally contain different amounts of the individual cannabinoids (Elsohly, M. A. and D. Slade (2005). “Chemical constituents of marijuana: the complex mixture of natural cannabinoids.” Life Sciences 78(5): 539-548), and to synthetic analogues of phytocannabinoids, which compounds may be manufactured by isolation from Cannabis plants and chemovars thereof, by using yeast or other means utilizing biotechnology, by chemical synthesis, by combination of these methods, or by any other means. The terms “cannabinoid” or “cannabinoids” refer to compounds having logP or clogP or wherein logP is an n-octanol/water partition coefficient obtained experimentally or calculated (clogP) by methods known to those skilled in the art. The terms “cannabinoid” or “cannabinoids” refer, therefore, for example, to (−)-trans-Δ⁹-tetrahydrocannabinol (Δ⁹-THC or THC), Δ⁸-tetrahydrocannabinol (Δ⁸-THC), (−)-trans-cannabidiol (CBD), cannabinol (CBN), cannabichromene (CBC), cannabicyclol (CBL), cannabielsoin (CBE), cannabinoldiol, cannabitriol, cannabigerol (CBG), cannabifuran (CBF), and their homologues containing a propyl rather than a pentyl side chain, such as cannabidivarin (CBDV), cannabivarin (CBV or cannabivarol), tetrahydrocannabivarin (THCV or THV), cannabichromene propyl analogue, as well as nabilone (racemic mixture or mixture of individual enantiomers in whatever stereoisomeric excess or purity), levonantradol (CP 50,556-1), cannabilactone (AM-1714), cannabicyclohexanol ((C8)-CP 47,497), (C9)-CP 47,497, AM-2389, AM-4030, AM-4056, (−)-HU-210, (+)-HU-210, racemic HU-210 or a mixture of individual enantiomers in whatever stereoisomeric excess or purity, ajulemic acid (HU-239), HU-243, HU-308, HU-320, HU-331, HU-336, HU-345, 11-hydroxy-Δ⁹-tetrahydrocannabinol (11-OH-THC), 11-carboxy-Δ⁹-tetrahydrocannabinol (11-CO₂H-THC), 7-hydroxycannabidiol (7-OH-CBD), 7-carboxycannabidiol (7-OH-CBD), CP 55,940, JWH-133, AM-087, AM-356, AM-404, AM-678, AM-855, AM-905, AM-906, AM-919, AM-938, CP 47,497, (C6)-CP 47,497, (C7)-CP 47,497, CP 55,940, AMG-36, AMG-41, KM-233, JWH-051, JWH-102, JWH-056, JWH-057, JWH-065, JWH-103, JWH-133, JWH-139, JWH-142, JWH-143, JWH-161, JWH-186, JWH-187, JWH-188, JWH-190, JWH-191, JWH-215, JWH-216, JWH-217, JWH-224, JWH-225, JWH-226, JWH-227, JWH-229, JWH-230, JWH-233, JWH-247, JWH-254, JWH-256, JWH-277, JWH-278, JWH-298, JWH-299, JWH-300, JWH-301, JWH-310, JWH-336, JWH-338, JWH-339, JWH-340, JWH-341, JWH-349, JWH-350, JWH-352, JWH-353, JWH-354, JWH-355, JWH-356, JWH-357, JWH-358, JWH-359, JWH-360, JWH-361, JWH-362, O-1871, and any combination of two or more of these compounds. Cannabinoids may be isolated from plants as mixtures of cannabinoids and other plant-derived materials, such as terpenes, flavonoids, etc. or cannabinoids may be purified substances, and may be amorphous or exist in one or more different crystalline states (polymorphs). See U.S. Pat. Nos. 7,169,942; 10,221,164; 7,169,942; 10,221,164; 7,759,526; 4,228,169; 7,179,800; and US Pat. Appln. Nos. 2006/0183922; 2005/000990; 2004/0087590.

The concentration of a cannabinoid, or a mixture of two or more cannabinoids, in a formulation may be approximately 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or 40 mg/ml. Alternatively, the concentration of a cannabinoid, or a mixture of two or more cannabinoids, in a formulation may be 1 mg/g, 2 mg/g, 3 mg/g, 4 mg/g, 5 mg/g, 6 mg/g, 7 mg/g, 8 mg/g, 9 mg/g, 10 mg/g, 11 mg/g, 12 mg/g, 13 mg/g, 14 mg/g, 15 mg/g, 16 mg/g, 17 mg/g, 18 mg/g, 19 mg/g, 20 mg/g, 21 mg/g, 22 mg/g, 23 mg/g, 24 mg/g, 25 mg/g, 26 mg/g, 27 mg/g, 28 mg/g, 29 mg/g, 30 mg/g, 31 mg/g, 32 mg/g, 33 mg/g, 34 mg/g, 35 mg/g, 36 mg/g, 37 mg/g, 38 mg/g, 39 mg/g, or 40 mg/g. Alternatively, a cannabinoid or a mixture of cannabinoids may be present in a weight to weight (w/w) ratio relative to phospholipid of 1/20, 1/19, 1/18, 1/17, 1/16, 1/15, 1/14, 1/13, 1/12, 1/11, 1/10, 1/9, 1/8, 1/7, 1/6, or 1/5.

In preferred embodiments, the cannabinoids are Δ⁹-THC, Δ⁸-THC, CBD, and CBN or mixtures thereof. In most preferred embodiments, the cannabinoid(s) is/are one or both of THC and CBD. In some embodiments, the cannabinoid is CBD (for example, cannabis-derived CBD or hemp-derived CBD). In some embodiments, the CBD is hemp-derived and contains less than 0.3% THC.

As used herein, the term “phospholipid” or “phospholipids” refers to amphiphilic compounds comprising at least one saturated or unsaturated hydrophobic fatty acid moiety and a hydrophilic moiety comprising a phosphate group. These include, for example, dicetyl phosphate, soya phosphatidylcholine (SPC), egg phosphatidylcholine (EPC), hydrogenated soya phosphatidylcholine (HSPC), soya lecithin, hydrogenated soya lecithin, sphingomyelin, dioleoyl phosphatidylcholine (DOPC), dilinoleoyl phosphatidylcholine (DLPC), dioleoyl phosphatidylethanolamine (DOPE), dimyristoyl phosphatidylethanolamine (DMPE), dipalmitoyl phosphatidylethanolamine (DPPE), dimyristoyl phosphatidylcholine (DMPC), dipalmitoyl phosphatidylcholine (DPPC), distearoyl phosphatidylcholine (DSPC), dilauroyl phosphatidylcholine (DLPC), 1-myristoyl-2-palmitoyl phosphatidylcholine, 1-palmitoyl-2-myristoyl phosphatidylcholine, 1-palmitoyl phosphatidylcholine, 1-stearoyl-2-palmitoyl phosphatidylcholine, dipalmitoyl sphingomyelin, distearoyl sphingomyelin, soya phosphatidylinositol (SPI), hydrogenated phosphatidylinositol (HPI), dimyristoyl phosphatidylglycerol (DMPG), dipalmitoyl phosphatidylglycerol (DPPG), distearoyl phosphatidylglycerol (DSPG), dimyristoyl phosphatidic acid (DMPA), dipalmitoyl phosphatidic acid (DPPA), dimyristoyl phosphatidylserine (DMPS), dipalmitoyl phosphatidylserine (DPPS), hydrogenated soya phosphatidylglycerol, dioleoyl phosphatidylglycerol (DOPG), distearoyl phosphatidic acid (DSPA), and mixtures thereof, and salts thereof, preferably sodium or ammonium salts. Phospholipids may be present, on weight-to-weight (w/w) basis relative to total weight of a composition, at a level of 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, or 25%. In preferred embodiments, the phospholipid is one of or is a combination of two or more of SPC, EPC, HSPC, or DSPC. In certain embodiments, the liposome constituent lipids do not include cholesterol or its derivatives. In some embodiments, the lipids consist of, or consist essentially of, of the phospholipids recited above, or a subset thereof.

As used herein, the term “cryoprotectant” or “cryoprotectants” or “bulking agent” or “bulking agents” refers to compounds such as, for example, mannitol, sorbitol, lactose, trehalose, sucrose, dextran of different molecular weights such as dextran 40, inulin, glycine, L-arginine, α-cyclodextrin, β-cyclodextrin, γ-cyclodextrin, hydroxypropyl-β-cyclodextrin, hydroxypropyl-γ-cyclodextrin, randomly methylated-β-cyclodextrin, sulfobutyl ether β-cyclodextrin (SBE β-CD), hydroxypropyl methylcellulose (HPMC, hypromellose), methylcellulose, polyvinylpyrrolidone (PVP) K15, K16-18, K30, or K90, citric acid, sodium citrate, poloxamer 188 (Pluronic® F-68), poloxamer 407 (Pluronic® F-127), or polyvinyl caprolactam-polyvinyl acetate-polyethylene glycol graft copolymer (Soluplus®) .

As used herein, the term “stabilizer” or “stabilizers” refers to, for example, ascorbic acid, ascorbate salts such as sodium or potassium ascorbate, citric acid, citrate salts such as, for example, sodium or potassium citrate, ethylenediaminetetraacetic acid (EDTA), EDTA salts such disodium EDTA, dipotassium EDTA, trisodium EDTA, tetrasodium EDTA, or calcium disodium EDTA, hydroxyethyl ethylenediamine triacetic acid (HEDTA), trisodium HEDTA, diethylenetriaminepentaacetic acid (DTPA), ethylenediamine-NN-disuccinic acid (EDDS), trisodium EDDS, DTPA pentasodium salt (pentasodium diethylenetriaminepentaacetate), methylglycinediacetic acid, trisodium dicarboxymethyl alaninate, d-glucono-1,5-lactone, gluconic acid and its salts such as sodium or potassium gluconate, or calcium gluconate, iminodisuccinic acid tetrasodium salt (tetrasodium iminodisuccinate), α-tocopherol, α-tocopherol acetate, ascorbyl palmitate, ascorbyl stearate, butylated hydroxytoluene (BHT), or butylated hydroxyanisole (BHA).

As used herein, the term “water-miscible solvent”, “water-miscible solvents”, “water-soluble solvent”, or “water-soluble solvents” refers to compounds such as, for example, ethyl alcohol (ethanol), t-butyl alcohol (t-butanol, tert-butanol, or TBA), polyethylene glycols (PEGs or macrogols) of different molecular weights such as PEG 300, PEG 400, PEG 600, PEG 1500, glycerin, diethylene glycol monoethyl ether (Transcutol®, diethylene glycol ethyl ether or 2-(2-ethoxyethoxy)ethanol), triacetin (glycerin triacetate), and propylene glycol (PG), which solvents may be used alone or as a combination of two or more solvents, with water-miscible solvents comprising, on weight-to-weight (w/w) basis relative to total weight of a formulation of 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%. In general, the compositions of the invention contain no more than 20% of PG, no more than 20% of glycerin, and no more than 20% of both PG and glycerin when both are present. Preferably, the compositions of the invention contain 6-20%, 8-18%, 6-16%, 6-14%, 8-16%, 8-14%, or 8-12% of PG. Likewise, the compositions of the invention contain 6-20%, 8-18%, 6-16%, 6-14%, 8-16%, 8-14%, or 8-12% of glycerin.

As user herein, the term “antimicrobial agent”, or “antimicrobial agents”, or “antimicrobial”, or “antimicrobials”, or “preservative”, or “preservatives” refers to substances that inhibit growth or kill microorganisms, whether antibacterial and/or antifungal agents, such as, for example, methyl paraben (methylparaben), ethyl paraben (ethylparaben), propyl paraben (propylparaben), butyl paraben (butylparaben), and heptyl paraben (heptylparaben), benzoic acid and benzoic acid salts such as sodium benzoate, dehydroacetic acid and sodium dehydroacetate, sorbic acid and its salts such as sodium sorbate, salicylic acid and its salts such as sodium salicylate, p-anisic acid, caprylhydroxamic acid, caprylic acid and its salts such as sodium caprate, levulinic acid and its salts such as sodium levulinate, undecylenic (10-undecenoic) acid and its salts such as sodium undecylenate, eugenol, menthol, 1,2-pentanediol, 1,2-hexanediol, 1,2-octanediol, 1,2-decanediol, ethylhexylglycerin, glyceryl caprate, glyceryl caprylate, glyceryl undecylenate, phenethyl alcohol, and phenylpropanol. The antimicrobial agents, whether used singly or as a blend of two or more antimicrobial agents, are to be used in the concentrations that vary from agent to agent and are to be introduced into the formulations in either organic or aqueous phase, all of which is known to those skilled in the art.

As used herein, the term “thickener” or “thickening agent” refers to substances, whether gelling or non-gelling, which raise viscosity and which may or may not require pH adjustment or addition of salts (ions) to produce increase in viscosity. Examples of “thickeners” or “thickening agents” are crosslinked polyacrylic acid polymers such as Carbopol® 71G, 940, 971P, 974P, 980, 981, 5984 EP, ETD 2020, Ultrez 10, Pemulen™ TR-1 and TR-2 NF polymers; hydroxyethyl acrylate/sodium acryloyldimethyl taurate copolymers; polyacrylate crosspolymer-6; sodium acrylate/acryloyldimethyltaurate/dimethylacrylamide crosspolymer; hyaluronic acid of average molecular weights of approximately 8,000-13,000, 50,000-75,000, 450,000-500,000, or one million or more Da; hydroxypropyl methylcellulose (HPMC, hypromellose, substitution types 2910, 2208, or 2906) in grades of viscosity of 2% aqueous solution of approximately 3 cP, 4 cP, 5 cP, 15 cP, 50 cP (40-60 cP), 100 cP (80-120 cP), 200-300 cP, 500-1000 cP, 1000-2000 cP, 4000 cP, methylcellulose, hydroxyethyl cellulose in grades of viscosity of 5% aqueous solution of 100 cP, 50-150 cP, of 2% aqueous solution at 20° C. of 200-300 cP, 800-1500 cP, approximately 2000 cP, approximately 3400 cP, or 5000 cP, ethylcellulose, hydroxypropyl cellulose, also gums such as xanthan gum, locust bean gum, guar gum, alginin, as well as agar gum, pectin, K-carrageenan, 1-carrageenan, as well as starches such as potato starch, corn (maize) starch, wheat starch, or pea starch. Some of the thickeners are multifunctional substances and in certain compositions a thickener may act as an anti-caking agent and/or a lubricating agent, and/or a humectant. As used herein, the term “lubricating agent” may refer to a thickener or it may refer to a substance that is not a thickener, for example, to lauric acid and its salts such as sodium laurate, or isopropyl myristate.

As used herein, the term “formulation” is used interchangeably with the term “composition.” In general, a “formulation” of the invention comprises one or more cannabinoids and phospholipids, and may contain one or more of surfactants, cryoprotectants, bulking agents, stabilizers, water-miscible solvents, anti-microbial agents, or thickeners. The compositions described herein are intended for use in pharmaceutical, phytopharmaceutical, nutraceutical, cosmetic, or veterinary settings by various routes of administration, such as dermal (topical or transdermal), mucosal (buccal, sublingual, gingival, vaginal, or rectal), or enteral (oral, ingestible) and may be formulated as an ointment, a cream, a suspension, a lotion, a paste, a gel, or a suppository, or in soft- or hard-shell capsules, or tinctures, or fluids of different viscosities, or serums, the basic preparation techniques of which are known to those skilled in the art.

As used herein, the term “application” or “applying”, or “administration”, or “administering” means placing or spreading or rubbing on a quantity of a composition to female subject's genital area(s), such as on or around external genitalia, for example, onto absorptive mucosa, comprising one or more of: the introitus, the vulva, the labia minora, the clitoris and the vaginal vault.

When desirable, appropriate preservatives may be added, such as anti-microbial and anti-fungal agents or other agents as described above.

The term “sexual activity” refers to sexual intercourse or other stimulation with a partner or masturbation. Formulation of the invention can be produced by a number of methods, including those described in the Examples and claims below.

The following examples are not intended to be limiting. Those of skill in the art will, in light of the present disclosure, appreciate that many changes can be made in the specific materials and which are disclosed and still obtain a like or similar result without departing from the spirit and scope of the invention.

### EXAMPLES

### Example 1. In Vitro Isometric Tension Experiments on Vaginal Tissue

The following in vitro methodology was utilized to examine the concentration-dependent response of rat vaginal tissue, both proximal and distal, to CBD following contraction by electrical field stimulation (similar to efferent neurostimulation in vivo)

### Tissue Preparation

Female rats either intact (n=6) or bilaterally oophorectomized (OVX) (n=6 rats); the animals were euthanized 6 weeks post OVX and vagina dissected. Then, the vagina was cut into 2 halves, proximal and distal, and 4 strips max per region cut in circular direction were prepared and mounted as shown in FIG. 1.

### Example 2. Evaluation of the Smooth Muscle Reactivity With Isolated Organ Baths

The strips were excised from the tissue samples and connected to force transducers for isometric tension recording. Organ baths were filled with Krebs buffer maintained at 37° C. and bubbled with 95% O₂ and 5% CO₂, pH 7.4.

TABLE 1
Evaluation of vaginal reactivity on electrical
field stimulation (EFS) - induced contraction
Preparation of vaginal strips (2 rats)

| Proximal vagina | | | | Distal vagina | | | |
|---|---|---|---|---|---|---|---|
| Strip | Strip | Strip | Strip | Strip | Strip | Strip | Strip |
| 3 | 3 | 4 | 4 | 3 | 3 | 4 | 4 |
| Rat 1 | Rat 2 | Rat 1 | Rat 2 | Rat 1 | Rat 2 | Rat 1 | Rat 2 |

Equilibration 60 min
Priming period: KCl
Wash
Priming period: EFS
Wash

| | | | |
|---|---|---|---|
| Time Control | Baseline | Time Control | Baseline |
| FRC | FRC | FRC | FRC |
| Time Control | Vehicle | Time control | Vehicle |
| FRC | Incubation | FRC | Incubation |
| | followed | | followed |
| | by a FRC | | by a FRC |

Wash

| | | | |
|---|---|---|---|
| Time control | CBD 1 μg/ml | Time Control | CBD 1 μg/ml |
| FRC | incubation | FRC | incubation |
| | followed | | followed |
| | by a FRC | | by a FRC |

Wash

| | | | |
|---|---|---|---|
| Time control | CBD 10 μg/ml | Time Control | CBD 10 μg/ml |
| FRC | incubation | FRC | incubation |
| | followed | | followed |
| | by an FRC | | by a FRC |

Wash

| | | | |
|---|---|---|---|
| Time control | CBD 100 μg/ml | Time control | CBD 100 μg/ml |
| FRC | incubation | FRC | incubation |
| | followed | | followed |
| | by an FRC | | by a FRC |

### Example 3. A Dose-Response (Concentration Response) Relaxation of Rat Vaginal Smooth Muscle Tissue to Cannabidiol Harvested from Rats With Intact Ovaries (“Premenopausal”)

Female rats without (n=6) prior bilateral oophorectomies were euthanized and the proximal and distal vaginal tissue was harvested and suspended in a classic organ bath infused at a physiological temperature, pH, and oxygen level. The untreated tissue was examined and confirmed to be responsive to electrical field stimulation (the in vivo equivalent of autonomic stimulation) and then the inhibition of the contraction response to a 1 Hz to 48 Hz range was observed in response to the addition of CBD in incremental doses of 1 μg/ml, 10 μg/ml and 100 μg/ml. CBD demonstrated dramatic and significant dose-response relaxation in non-oophorectomized rats. This was also true regardless of whether the tissue was from the proximal or distal vaginal tissue (FIG. 2(A-E)).

### Example 4. A Dose-Response (Concentration Response) Relaxation of Rat Vaginal Smooth Muscle Tissue to Cannabidiol Harvested from Rats With Bilateral Oophorectomies (“Postmenopausal”)

Female rats with (n=6) prior bilateral oophorectomies were euthanized and the proximal and distal vaginal tissue was harvested and suspended in a classic organ bath infused at a physiological temperature, pH, and oxygen level. The untreated tissue was examined and confirmed to be responsive to electrical field stimulation (the in vivo equivalent of autonomic nerve stimulation) and then the inhibition of the contraction response to a 1 Hz to 48 Hz range was observed in response to the addition of CBD in incremental doses of 1 μg/ml, 10 μg/ml and 100 μg/ml, CBD demonstrated dramatic and significant dose-response relaxation in oophorectomized rats. This was also true regardless of whether the tissue was from the proximal or distal vaginal tissue (FIG. 3(A-E)).

These effects, for the first time, demonstrate that cannabidiol has an unexpected, peripheral, dose-dependent pharmacological relaxant effect on rat vaginal smooth muscle tissue, regardless of the female hormone status (FIG. 4 and FIG. 5). This implies that a pharmacologic response should be seen in both premenopausal and postmenopausal women. This smooth muscle relaxation in the vagina and clitoris is a prerequisite for vaginal engorgement and lubrication as well as clitoral engorgement and elongation which in turn are the hallmark of sexual arousal in women.

### Conclusions

1. CBD at 1, 10 and 100 μg/ml showed a significant relaxant effect on EFS-induced contractions of vaginal strips from intact/sham and oophorectomized rats compared to vehicle.
2. Furthermore, CBD exhibited its relaxant effect in a concentration-response manner on vagina strips from intact/sham and oophorectomized rats, whether distal or proximal strips.

### Example 5. One-Pot Preparation of CBD-Loaded PG Liposomes

Hydrogenated soya phosphatidylcholine (5.4 grams) and CBD (0.6 grams) were dissolved in propylene glycol (6 mL) in a closed vessel by heating in a water bath at approximately 85° C. with magnetic stirring. This solution was added quickly, with overhead stirring, to a solution of citric acid (54 mg) and sodium ascorbate (600 mg) in 50 mL of deionized water pre-warmed in a water bath at 60° C. to form a white suspension. The suspension was stirred at 60° C. bath temperature for approximately one hour then removed from heat with continued stirring.

### Example 6. One-Pot Preparation of Lotion of CBD-Loaded PG Liposomes

Hydrogenated soya phosphatidylcholine (10.8 grams) and CBD (1.2 grams) were dissolved in propylene glycol (12 mL) by heating in a water bath at 80-90° C. with magnetic stirring. This solution was added, with overhead stirring, over approximately 30 seconds to a solution of ascorbic acid (50 mg) and sodium ascorbate (500 mg) in 100 mL of deionized water pre-warmed in a water bath at 65° C. to form a white suspension. The suspension was stirred at 65° C. bath temperature for approximately 30 minutes then removed from heat with continued stirring. To a warm suspension, with continued stirring, was added 300 mg (0.25% w/w) polyacrylate crosspolymer-6 to form a white lotion.

### Example 7. One-Pot Preparation of CBD-Loaded Glycerosomes

Hydrogenated soya phosphatidylcholine (2.7 grams), CBD (0.3 grams), and polyethylene glycol monostearate (50 mg) were mixed with glycerin (3 mL) and deionized water (27 mL) containing 25 mg citric acid and 275 mg sodium ascorbate, and heated in a water bath at 80° C. with magnetic stirring until a homogenous suspension was formed. Evaluation by optical microscopy revealed presence of a mixture of round vesicles and vesicle aggregates approximately 1-10 pm in size.

### Example 8. Physiologic Studies in Women—Vaginal Photoplethysmography Demonstration of Increased Vaginal Blood Flow Following the Administration of the Invention

According to the study design, three young (ages 20-30) women undergo in-lab, standard vaginal photoplethysmography studies examining the blood flow responses, including vaginal pulse amplitude (VPA) and photographs of vaginal perfusion, EEG alpha-wave activity, and galvanic skin resistance (GSK) measurements. Measurements are made at baseline and 30 minutes following application of the invention and in the presence of visual sexual stimulation. If results are positive, they indicate that the invention is able to increase vaginal blood flow and results in an increase in subjective and objective measurements of sexual arousal.

### Example 9. Studies in Women At-Home, Utilizing a Comprehensive Sexual Function Questionnaire

Twenty female volunteers, ages 21-65 years, participated in an at-home sexual response study utilizing the invention. They had normal or nearly normal sexual interest, normal or nearly normal arousal (clitoral engorgement and vaginal lubrication) and had the ability to orgasm at least some time with a partner or with masturbation. They received four doses of the composition of invention, identical to, or very similar to the lotion described in Example 6. Each dose was applied by hand to the labia minora (inner lips), vulva (outside of vagina and inside of labia minora) and clitoris, 20 minutes prior to partner or self-stimulation. Each participant made 4 attempts using the invention at intervals no more than once per day and all 4 attempts were completed within 4 weeks. Following the completion of the 4 episodes, the volunteers completed an online questionnaire, which was structured to include the elements of the Female Sexual Function Index (Rosen) and examined desire/interest, arousal, orgasm, overall sexual satisfaction and any personal perspectives on the invention and its effect on their sexual function. Reports of adverse events were also captured. The questionnaire is exemplified in Appendix A.

### APPENDIX A: Manna™ Female Sexual Response Questionnaire© 2019

1. Over the past 4 weeks, when you used the study product, how would you rate your level (degree) of sexual desire or interest?
A. Higher than usual
B. As usual
C. Less than usual

2. Over the past 4 weeks, when you used the study product, how often did you become lubricated (“wet”) during sexual activity?
A. More often than usual
B. As usual
C. Less often than usual

3. Over the past 4 weeks, when you used the study product, how often did you maintain your lubrication (“wetness”) until completion of sexual activity?
A. More often than usual
B. As usual
C. Less often than usual

4. Over the past 4 weeks, when you used the study product and had sexual stimulation, how often did you reach orgasm (climax)?
A. More often than usual/Most or all the time
B. As usual
C. Less often than usual

5. Over the past 4 weeks, when you used the study product and had sexual stimulation, was the ease of achieving your orgasms:
A. Easier
B. As usual
C. More difficult

6. Over the past 4 weeks, when you used the study product and had sexual stimulation, was the intensity of your orgasms:
A. More intense
B. As usual
C. Less intense

7. Over the past 4 weeks, when you used the study product, how satisfied were you with your ability to reach orgasm (climax) during sexual activity or intercourse?
A. More satisfied than usual
B. As satisfied as usual
C. Less satisfied than usual

8. Over the past 4 weeks, how satisfied have you been with your overall sexual life?
A. More satisfied than usual
B. As satisfied as usual
C. Less satisfied than usual

9. Based on your experience with the study product, how often would you use the product once it became commercially available?
A. Every time I engage in sexual activity
B. Some of the time I engage in sexual activity
C. Once in a while
D. Never

### Example 10. Studies in Women At-Home, Utilizing a Comprehensive Sexual Function Questionnaire—Aggregate Responses of the Study Described in Example 9

The responses in the questionnaires completed by all 20 participants referred to in Example 9 were combined and grouped into domains of Desire (Question 1), Arousal (Questions 2 and 3), and Orgasm (Questions 4, 5, 6 and 7). The aggregated results per category were: Increased Desire was reported by 7 out of 20 participants or 35%; Increased Arousal—frequency and/or ease of lubrication was reported by 12 out of 20 participants or 60%; Orgasm—frequency, ease, intensity and/or satisfaction was reported by 18 out of 20 participants or 90%.

Overall outcomes of this real-world, at-home study: the positive outcomes in this patient population with intact sexual function is surprising since they had a narrower range to move upward. These outcomes point to a treatment value in women with overt dysfunction/disorder in interest/arousal and/or orgasms. Additionally, women with genitopelvic pain due to inadequate lubrication or post-menopausal vaginal atrophy are expected to respond as well.

### Example 11. At-Home, Self-Reported Effects on Sexual Function in a Young (<30 Years of Age), Motivated Volunteer

The participant was one of the 20 volunteers referred to in Example 9 and the study was conducted as described therein.

A young pre-menopausal woman reported the following positive outcomes:

1. Over the 4 weeks, when she used the invention, she became lubricated (“wet”) more often during sexual activity;

2. Over the past 4 weeks, when she used the invention, she maintained lubrication (“wetness”) more often during sexual activity;

3. Over the past 4 weeks, when she used the invention and had sexual stimulation, she reached orgasm (climax) more often/most of the time than usual;

4. Over the past 4 weeks, when she used the invention and had sexual stimulation, she experienced greater ease of achieving orgasms than usual;

5. Over the past 4 weeks, when she used the invention, she was more satisfied with her ability to reach orgasm (climax) during sexual activity or intercourse than usual;

6. No adverse events were reported.

### Example 12. At-Home, Self-Reported Effects on Sexual Function in a Young (<30 Years of Age), Motivated Volunteer

The participant was one of the 20 volunteers referred to in Example 9 and the study was conducted as described therein.

A young, pre-menopausal woman reported the following positive outcomes:

1. Over the past 4 weeks, when she used the study product, she reported a higher level (degree) of sexual desire or interest, than usual;

2. Over the past 4 weeks, when she used the study product and had sexual stimulation, she reached orgasm more often/ most of the time, than usual;

3. Over the past 4 weeks, when she used the study product and had sexual stimulation, she experienced greater ease of achieving orgasms;

4. No adverse events.

### Example 13. At-Home, Self-Reported Effects on Sexual Function in a 60 Year Old, Post-Menopausal Motivated Volunteer

The participant was one of the 20 volunteers referred to in Example 9 and the study was conducted as described therein.

A 60-year old, postmenopausal woman reported the following positive outcomes:

1. Over the past 4 weeks, when she used the study product, she reported a higher level (degree) of sexual desire or interest, than usual;

2. Over the past 4 weeks, when she used the study product and had sexual stimulation, she experienced more intense orgasms;

3. Over the past 4 weeks, when she used the study product and had sexual stimulation, she was more satisfied with her ability to reach orgasm (climax) during sexual activity or intercourse than usual;

4. No adverse events.

### Example 14. At-Home, Self-Reported Effects on Sexual Function in a Young (33 Years of Age), Motivated Volunteer

The participant was one of the 20 volunteers referred to in Example 9 and the study was conducted as described therein.

A young pre-menopausal woman reported the following positive outcomes:

1. Over the 4 weeks, when she used the invention, she became lubricated (“wet”) more often during sexual activity;

2. Over the past 4 weeks, when she used the invention, she maintained lubrication (“wetness”) more often during sexual activity;

3. Over the past 4 weeks, when she used the invention and had sexual stimulation, she experienced greater ease of achieving orgasms than usual;

4. Over the past 4 weeks, when she used the invention, she was more satisfied with her ability to reach orgasm (climax) during sexual activity or intercourse than usual;

5. No adverse events were reported.

### Example 15. At-Home, Self-Reported Effects on Sexual Function in a Post-Menopausal 61 Year Old, Motivated Volunteer

The participant was one of the 20 volunteers referred to in Example 9 and the study was conducted as described therein.

A 61-year old, postmenopausal woman reported the following positive outcomes:

1. Over the past 4 weeks, when she used the study product, she reported a higher level (degree) of sexual desire or interest, than usual;

2. Over the past 4 weeks, when she used the invention, she maintained lubrication (“wetness”) more often during sexual activity;

3. Over the past 4 weeks, when she used the study product and had sexual stimulation, she experienced more frequent orgasms;

4. Over the past 4 weeks, when she used the invention and had sexual stimulation, she experienced greater ease of achieving orgasms than usual;

5. Over the past 4 weeks, when she used the study product and had sexual stimulation, she was more satisfied with her ability to reach orgasm (climax) during sexual activity or intercourse than usual;

6. No adverse events.

### Example 16. At-Home, Self-Reported Effects on Sexual Function in a 27 Year Old Woman Comparing the Invention to a CBD Tincture

The participant was not one of the 20 volunteers referred to in Example 9. The study was conducted as described therein except for the subsequent evaluation of the effects of an orally taken CBD tincture (Bluebird Botanicals, Classic).

A 27-year old woman with anorgasmia underwent testing of the product for 4 weeks with 4 episodes of sexual activity following application of the product. She reported the following positive outcomes:

1. Over the past 4 weeks, when she used the study product, she reported a higher level (degree) of sexual desire or interest, than usual;

2. Over the past 4 weeks, when she used the study product and had sexual stimulation, she experienced greater ease of achieving orgasms;

3. Over the past 4 weeks, when she used the study product and had sexual stimulation, she experienced more intense orgasms;

4. No adverse events.

The volunteer then underwent a 1 week washout period and subsequently the same self-administered questionnaire based on the Female Sexual Function Index questionnaire recorded at-home responses following four separate sexual events that were preceded by the self-administration of approximately 21 mg of hemp CBD tincture one hour prior to sexual activity. The woman reported no change in the baseline levels of desire, arousal, and remained anorgasmic. She had no adverse events with the use of the tincture.

The results of this study indicate that the observed effects of the invention are specific to the invention and its method application. That is, the positive effects observed with the at-home studies mean that a sufficiently high CBD dose is delivered to the vaginal and clitoral smooth muscle from local application of the invention, thereby effecting greater smooth muscle relaxation and greater impact on arousal and orgasm than is possible by ingestion of a comparable dose of CBD.

### Example 17. Facilitating Safe Sexual Practice—Condom-Compatibility Testing

Following the general principles of the American Society for Materials Testing, the tensile strength and air burst capacity of latex and polyisoprene condoms were examined following the application of the invention and compared it to non-encapsulated CBD. In a control experiment, exposure of a stretched latex condom (Durex®) to t-butanol had no effect but exposure of a stretched latex condom (Durex®) to a 10 mg/ml solution of CBD in t-butanol (non-encapsulated CBD) caused instant rupture. In contrast, the latex condoms and polyisoprene condoms demonstrated preserved tensile strength and preserved burst/rupture characteristics of the native control condoms. Testing involved tensile and air burst testing on three different brands of latex condoms, and one brand of polyisoprene condoms. Each brand of condoms was from a single, finished lot.

These study results also point to the safe combination of the invention as a lubricant in combination with a condom as an alternative route to administration.

### Example 18. Facilitating Safe Sexual Practice—Condom-Compatibility Testing by a specialized Testing Laboratory

The compatibility testing was conducted in accordance with:

ASTM D7661-18: Standard Test Method for Determining Compatibility of Personal Lubricants with Natural Rubber Latex Condoms; ASTM D3492-16: Standard Specification for Rubber Contraceptives (Male Condoms).

The testing was performed on three types of latex condoms: Trojan Enz non-lubricated latex, Lifestyles non-lubricated latex, and Atlas non-lubricated latex; one type of polyisoprene condom: Lifestyles SKYN® lubricated polyisoprene; and one type of polyurethane condom: Trojan® Supra lubricated polyurethane.

The test product application, removal, and the condom compatibility testing were performed per instructions described in ASTM D7661-18. No modifications were employed. A minimum of 20 samples per condom were prepared for testing as follows:

Not exposed to lubricant or conditioned (Baseline);

Conditioned, but not exposed to lubricant (Control);

Exposed to the subject lubricant/sample and conditioned (Subject Lubricant);

Exposed to a Positive Control (mineral oil) and conditioned (Positive Control).

Conditioning was performed by exposing the materials at 40° C. for 60 minutes in environmental chamber capable of maintaining 40±2° C.

The following conclusions were reached:

The mean change between the material from test sample and Trojan non-lubricated latex, Lifestyles non-lubricated latex, Atlas non-lubricated latex, and Lifestyles SKYN® polyisoprene male condoms was found to be <10% for break force, elongation, burst pressure, and burst volume.

The mean change between the material from test sample and Trojan Supra polyurethane male condoms was found to be <10% for elongation, and >20% for force break, burst pressure, and burst volume.

According to FDA guidance, the material from test sample is considered compatible with non-lubricated latex and polyisoprene male condoms, and non-compatible with polyurethane male condoms.

### Example 19. Treatment of Female Sexual Disorder, Including Sexual Interest/Arousal Disorder (SIAD), Female Orgasmic Disorder

The Diagnostic and Statistical Manual of Mental Disorders (“DSM”) includes the following two categories: (1) Sexual Interest/Arousal Disorder and (2) Female Orgasmic Disorder. These are common (affecting upwards of 40% of women) and known to significantly reduce the quality of life for women and their partners.

The current treatments for Interest/Arousal disorders include hormone replacement therapy and possibly androgen therapy. The latter is associated with such side effects as hirsutism and masculinization. More specific treatments also include flibanserin (Addyi®), which was originally developed as an antidepressant, flibanserin is approved by the Food and Drug Administration as a treatment for low sexual desire in premenopausal women but is associated with low efficacy and significant side-effects including low blood pressure, sleepiness, nausea, fatigue, dizziness and fainting, particularly if the drug is mixed with alcohol. Recently, the FDA approved bremelanotide (Vylessi®) for hypoactive sexual desire disorders (the prior classification that is included in the current Sexual Interest/Arousal Disorder category). Its mechanism of action is unknown but it works through melanocortin receptors in the CNS. However, it is administered through a subcutaneous injection 45 minutes prior to sexual activity. It too is associated with poor efficacy and side-effects are common, including nausea (in 40%) and vomiting, flushing, and headaches as well as injection site reactions.

There is a clear and, to date, an unmet need for an effective, peripherally acting formulation that can be utilized in a non-invasive, on-demand manner with few side-effects and those that do exist are not serious. The invention has demonstrated the ability to increase interest/arousal by a direct pharmacologic action and will improve both interest and arousal disorders as demonstrated in the prior examples when there is not an underlying medical or drug related side effect. It would be the first treatment for this disorder that is not a centrally acting drug and one that can be utilized on a prn or as needed basis to improve not just desire but also arousal. To date, there are no broadly effective or approved treatments for female arousal disorder. While drugs for male erectile dysfunction (a form of arousal disorder in men) such as sildenafil (^{Viagra}®) are effective in some women with arousal disorders secondary to anti-depressants, the efficacy rate is low and as such, the class of PDE5 inhibitors have not been approved for or utilized for female arousal disorder.

Finally, the ultimate outcome for women is a product that not only increases interest and/or arousal but also results in orgasm. The formulations of the invention are therefore expected to be effective in female interest/arousal disorder and female orgasmic disorder, unlike the other aforementioned therapies.

The composition of the invention may be tested in clinical trials for the treatment of female interest/arousal disorder and for female orgasmic disorder, for example, as follows:

Study 1: Inclusion of women, 18-70 years of age, with primarily interest/arousal disorder;

Study 2: Inclusion of women, 18-70 years of age, with interest/arousal intact but with orgasmic disorder.

Study design: Placebo-controlled, double-blind, randomized clinical trial with 4-week run-in period (and baseline FSFI measure) and then 3 months of treatment. The product is utilized on a prn basis, 20-30 minutes prior to sexual activity. Primary outcome measure: Female Sexual Function Index (FSFI) administered at the end of the study period.

The compositions of the invention are expected to increase the sexual interest/arousal domains by 1.5-2 in the first study and a similar 1.5-2 shift in the orgasm domain of the FSFI.

Female genitopelvic pain during or following intercourse resulting from inadequate lubrication (both premenopausal and post-menopausal) is expected to respond to formulations of the invention as well.

1-29. (canceled)

30. A method of improving female sexual function or treating a female sexual disorder, the method comprising applying a topical composition to a female subject's genital (arousal) area(s), wherein said topical composition formulated with liposomes that comprise cannabidiol (CBD) and wherein the CBD-containing composition is applied to mucosal surface in the amount and for a period of time prior to a sexual activity such that sexual function of the subject is enhanced or the disorder is ameliorated during the sexual activity.

31. The method of claim 30, wherein the topical composition comprises one or more additional cannabinoids.

32. The method of claim 30, wherein the genital area includes genitalia with absorptive mucosa, comprising one or more of: the introitus, the vulva, the labia minora, the clitoris and the vaginal vault.

33. The method of claim 30, wherein the enhancement of the sexual function and/or amelioration of the disorder is exhibited by one or both of the following parameters:
i. vaginal and clitoral smooth muscle relaxation; and
ii. increased vaginal and clitoral blood flow.

34. The method of claim 30, wherein the enhancement of the sexual function and/or amelioration of the disorder is exhibited by one or more of the following subjectively self-reported outcomes including:
i. “increased lubrication/wetness during sexual activity”;
ii. “reaching orgasm more often”;
iii. “greater ease or achieving orgasm”;
iv. “being more satisfied”;
v. “higher level of sexual desire”; and
vi. “reduction in pain during sexual activity”.

35. The method of claim 30, wherein the subject is premenopausal.

36. The method of claim 33 or 34, wherein the outcomes are reported by post-menopausal women are similar to those reported by premenopausal women with respect to one or more of the following parameters:
i. improved arousal;
ii. improved lubrication; and
iii. improved ease of achieving orgasm.

37. The method of claim 30, wherein the total amount of CBD applied is from 5 mg to 1,000 mg of CBD.

38. The method of claim 30, wherein the total amount of CBD applied is from 10-100 mg of CBD.

39. The method of claim 30, wherein the total amount of CBD applied is from 20-40 mg of CBD.

40. The method of claim 30, wherein the CBD-containing composition is applied at 5-30 mins prior to the sexual activity.

41. The method of claim 31, where the additional cannabinoid(s) is/are selected from the group consisting of Δ⁹-THC, Δ⁸-THC, CBD, and CBN.

42. A method of claim 30, wherein the CBD is hemp-derived and contains less than 0.3% THC by weight.

43. The method of claim 30, wherein the CBD-containing composition is provided in a single-use container.

44. The method of claim 30, wherein the CBD-containing composition comprises preservative(s) and is provided in a multi-use container.

45. The method of claim 30, wherein the CBD-containing composition is compatible with latex and polyisoprene condoms.

46. The method of claim 30, wherein the CBD-containing composition is provided by means of a condom pre-coated with the composition.

47. The method of claim 30, wherein the concentration of CBD in the composition is from 1 mg/ml to 40 mg/ml.

48. The method of claim 30, wherein the concentration of CBD in the composition is from 5 mg/ml to 20 mg/ml.

49. The method of claim 30, wherein the concentration of CBD in the composition is 10 mg/ml.

50. The method of claim 30, wherein the CBD-containing composition comprises liposomes that comprise HSPC, ascorbic acid, sodium ascorbate, propylene glycol, polyacrylate crosspolymer-6, and water or aqueous buffer, and wherein the liposomes are provided in a homogeneous suspension.

51. The method of claim 30, wherein the composition is applied 1-60 min prior to sexual activity.

52. The method of claim 30, wherein the composition is applied 5-40 min prior to sexual activity.

53. The method of claim 30, wherein the composition is applied 15-20 min prior to sexual activity.

54. The method of claim 30, wherein the subject is afflicted with a disorder selected from (1) Sexual Interest/Arousal Disorder (SIAD) and (2) Female Orgasmic Disorder.

55. The method of claim 54, wherein the subject, upon having been treated with the CBD-containing composition for 3-6 months, reports arousal and/or orgasmic improvements as measured by FSFI.

56. The method of claim 54, wherein the improvement in sexual interest/arousal domain is by 1.5-2 points, and by 1.5-2 points in the orgasm domain of the FSFI.

57. The method of claim 30, wherein the off-set time following the application of the CBD-containing composition is 0.5-5 hours.

58. The method of claim 30, wherein the off-set time following the application of the CBD-containing composition is 1-3 hours.

59. The method of claim 30, wherein the off-set time following the application of the CBD-containing composition is 1-2 hours.

60. The method of claim 30, wherein the CBD-containing composition comprises a phosphodiesterase type 5 inhibitor.

61. The method of claim 60, wherein the inhibitor is chosen from sildenafil, tadalafil, vardenafil, and udenafil.

62. The method of claim 30, wherein the CBD-containing composition comprises another direct smooth muscle relaxant.

63. The method of claim 62, wherein the relaxant is chosen from prostaglandin El, papaverine, and minoxidil.

64. The method of claim 30, wherein the CBD-containing composition comprises an alpha-blocker.

65. The method of claim 64, wherein the alpha-blocker is phentolamine.

66. The method of claim 30, wherein the CBD-containing composition comprises flibanserin or bremelanotide thereby augmenting sexual desire.
